(19) United States
(12) Patent Application Publication
(10) Pub. No.: US 2023/0317259 A1
(43) Pub. Date: Oct. 5, 2023

(54) SYSTEMS AND METHODS FOR VALIDATING HEALTHCARE REVIEWS VIA MEDICAL STAFF EXPERIENCE RECORDS

(71) Applicant: KONINKLIJKE PHILIPS N.V., EINDHOVEN (NL)

(72) Inventors: Prescott Peter Klassen, Cambridge, MA (US); Vincentius Paulus Buil, Eindhoven (NL); Mark Thomas Johnson, Eindhoven (NL); Murtaza Bulut, Eindhoven (NL); Alexander Sebastian Furnica, Eindhoven (NL)

(21) Appl. No.: 18/127,715

(22) Filed: Mar. 29, 2023

Related U.S. Application Data
(60) Provisional application No. 63/324,693, filed on Mar. 29, 2022.

(51) Int. Cl. G16H 40/20; G16H 10/60; G06Q 30/0282

(52) U.S. Cl. CPC G16H 40/20; G06Q 30/0282; G16H 10/60

(57) ABSTRACT

The present disclosure relates to a method for validating a healthcare review. The method includes: obtaining from an author, a request to post a healthcare review on a staff experience record; analyzing the healthcare review to identify a subject of the healthcare review; ascertaining identity of the author as to how the author is related with the subject; validating the healthcare review based on the identity of the author, at least one of personal health records and electronic medical records corresponding to the author or the subject, and the staff experience record; and determining a review validity score of the healthcare review based on results from the validating of the healthcare review.

### FIELD OF THE DISCLOSURE

The present disclosure relates to content validation of healthcare reviews, and more specifically, to computer-implemented systems and methods for numerically assessing and informing the validity of healthcare reviews.

### BACKGROUND

Online reviews provide a means to evaluate people, products, and service offerings. In the healthcare context, review sites provide evaluative information on hospitals, physicians, treatments, medication, etc. For example, healthcare review sites may offer reviews by patients about therapies, medication, surgery, and the like, while consumer review sites may assist in finding doctors, dentists, and other medical professionals. However, it is important that reviews be trusted. And particularly in the healthcare context, it is critical that reviews are a true reflection of the actual patient-experienced interactions with the care provided by the medical professionals. Further, the reviews should be a valid reflection of what can be expected by the patient, in relation to therapy adherence of the patient and the patient’s medical condition.

### SUMMARY OF THE DISCLOSURE

In accordance with aspects of the present disclosure, a computer implemented method includes: obtaining, by one or more processors, from an author, a request to post a healthcare review on a staff experience record; analyzing, by the one or more processors, the healthcare review to identify a subject of the healthcare review; ascertaining, by the one or more processors, an identity of the author as to how the author is related with the subject; validating, by the one or more processors, the healthcare review based on the identity of the author, at least one of personal health records and electronic medical records corresponding to a patient or the subject, and the staff experience record; determining, by the one or more processors, a review validity score of the healthcare review based on results from the validating of the healthcare review; and transmitting, by the one or more processors, the healthcare review and the validity score to the staff experience record.

In an aspect, the method also includes: where the step of ascertaining includes determining that the identity of the author is the patient who received a therapy or a person involved with providing care to the patient; and where the step of validating further includes determining a therapy adherence by the patient indicating how well the patient participated in the therapy, based on at least one of the personal health records and the electronic medical records of the patient, and one or more staff experience records of one or more healthcare professionals providing the therapy.

In an aspect, the method also includes: where the step of ascertaining includes determining that the identity of the author is the patient who received a therapy or a person involved with providing care to the patient; and where the step of validating further comprises determining a therapy effect coefficient indicating to what extent content of the healthcare review was caused by the therapy by use of therapy causation modeling based on at least one of the personal health records and the electronic medical records of the patient, one or more staff experience records of one or more healthcare professionals providing therapy, and one or more known therapy causation models.

In an aspect, the method also includes: where the step of ascertaining includes determining that the identity of the author is the patient who received a therapy or a person involved with providing care to the patient; and where the step of validating further comprises determining a certainty indicator corresponding to a therapy effect coefficient indicating how statistically reliable a therapy coefficient is.

In an aspect, the method also includes: where the step of ascertaining includes determining that the identity of the author is the patient who received a therapy or a person involved with providing care to the patient; and where the step of determining the review validity score comprises combining a therapy adherence, a therapy effect coefficient, and a certainty indicator by use of a selected statistical method to indicate how valid the healthcare review by the patient is in view of all available data.

In an aspect, the method also includes: where the step of ascertaining includes determining that the identity of the author is the patient who received a therapy or a person involved with providing care to the patient; and where the step of validating further includes determining a therapy effect coefficient indicating to what extent content of the healthcare review would have been caused by the therapy by use of therapy causation modeling based on at least one of the personal health records and the electronic medical records of the patient, one or more staff experience records of one or more healthcare professionals providing the therapy, and one or more known therapy causation models, where the therapy causation modeling comprises profiling a time progression of the subject in the healthcare review.

In an aspect, the method also includes: where the step of ascertaining includes determining that the identity of the author is the patient who received a therapy or a person involved with providing care to the patient; where the step of validating further comprises determining a therapy effect coefficient indicating to what extent content of the healthcare review would have been caused by the therapy by use of therapy causation modeling based on a combination of the personal health records or electronic medical records of the patient, one or more staff experience records of one or more healthcare professionals providing the therapy, and one or more known therapy causation models, where the therapy causation modeling includes profiling a timeline of the therapy for the patient.

In an aspect, the method also includes: where the step of ascertaining includes determining that the identity of the author is the patient who received a therapy or a person involved with providing care to the patient; where the step of validating further includes determining a therapy effect coefficient indicating to what extent content of the healthcare review would have been caused by the therapy by use of therapy causation modeling based on a combination of the personal health records or electronic medical records of the patient, one or more staff experience records of one or more healthcare professionals providing the therapy, and one or more known therapy causation models, where the therapy causation modeling includes profiling any time segments on effects of the therapy presented in a therapy causation model corresponding to the therapy.

In an aspect, the method also includes: where the step of ascertaining includes determining that the identity of the author is the patient who received a therapy or a person involved with providing care to the patient; where the step of validating further includes determining a therapy effect coefficient indicating to what extent content of the healthcare review would have been caused by the therapy by use of therapy causation modeling based on a combination of the personal health records or electronic medical records of the patient, one or more staff experience records of one or more healthcare professionals providing the therapy, and one or more known therapy causation models, where the therapy causation modeling includes analyzing the time progression of the subject, the respective times of the changes in the lifestyle, behavior, environment, or context of the patient, and the timeline of the therapy based on the time segments of the effect of the therapy.

In an aspect, the method also includes: where the step of ascertaining includes determining that the identity of the author is the patient who received the therapy or a person involved with providing care to the patient; where the step of validating further includes determining a therapy effect coefficient indicating to what extent content of the healthcare review would have been caused by the therapy by use of therapy causation modeling based on a combination of the personal health records or electronic medical records of the patient, one or more staff experience records of one or more healthcare professionals providing the therapy, where the therapy causation modeling includes: profiling a time progression of the subject in the healthcare review, respective times of any changes in a lifestyle, behavior, environment, or context of the patient that can affect the therapy with respect to the time progression, a timeline of the therapy for the patient, any time segments on effects of the therapy presented in a therapy causation model corresponding to the therapy; and analyzing the time progression of the subject, the respective times of the changes in the lifestyle, behavior environment, or context of the patient, and the timeline of the therapy based on the time segments of the effect of the therapy.

In an aspect, the method also includes: where the step of ascertaining includes determining that the identity of the author is the patient who received the therapy or a person involved with providing care to the patient; and where the step of validating further includes determining a therapy adherence by the patient indicating how well the patient participated in the therapy, a therapy effect coefficient indicating to what extent content of the healthcare review would have been caused by the therapy by use of therapy causation modeling, and a certainty indicator corresponding to the therapy causation coefficient indicating how statistically reliable the therapy coefficient is, based on a combination of the personal health records or electronic medical records of the patient, one or more staff experience records of one or more healthcare professionals providing the therapy, one or more known therapy causation models.

In an aspect, the method also includes: where the step of ascertaining includes determining that the identity of the author is the patient who received a therapy or a person involved with providing care to the patient; where the step of validating further includes: determining a therapy adherence by the patient indicating how well the patient participated in the therapy, based on personal health records or electronic medical records of the patient and staff experience records of a healthcare professional providing the therapy to the patient; determining a therapy effect coefficient indicating to what extent content of the healthcare review would have been caused by the therapy by use of therapy causation modeling based on a combination of the personal health records or electronic medical records of the patient, the staff experience records of the healthcare professional, and one or more known therapy causation models, where the therapy causation modeling includes analyzing a time progression of the subject, respective times of the changes in a lifestyle, behavior, environment, or context of the patient, and a timeline of the therapy based on the time segments of the effect of the therapy; and determining a certainty indicator corresponding to the therapy causation coefficient indicating how statistically reliable the therapy coefficient is; and where the step of determining the review validity score including: combining the therapy adherence, the therapy causation coefficient, and the certainty indicator by use of a selected statistical method to indicate how valid the healthcare review by the patient is in view of all available data.

In an aspect, the method also includes: where the step of ascertaining includes determining that the identity of the author is another healthcare professional representing an individual or an institute other than a healthcare professional providing therapy to the patient; and where the step of validating further includes probing a relationship between the author and the healthcare professional, based on respective staff experience records of the author or an employee record of the author to see if the relationship is likely to bias the healthcare review.

In an aspect, the method also includes: where the step of ascertaining includes determining that the identity of the author is another healthcare professional representing an individual or an institute other than a healthcare professional providing therapy to the patient; and where the step of validating further includes evaluating the healthcare review based on a standard operating procedures (SOP), care protocols, or guidelines regarding the therapy.

In an aspect, the method also includes: where the step of ascertaining includes determining that the identity of the author is another healthcare professional representing an individual or an institute other than a healthcare professional providing therapy to the patient; and where the step of validating further includes substantiating the healthcare review based on records from standard documentation practice regarding the therapy including the electronic medical records corresponding to the therapy, data logs from local or remote computer applications used in the therapy, or data logs from medical equipment used in the therapy.

In an aspect, the method also includes: where the step of ascertaining includes determining that the identity of the author is another healthcare professional representing an individual or an institute other than a healthcare professional providing therapy to the patient; and where the step of determining the review validity score includes combining the relationship between the author and the healthcare professional and respective results from the evaluating and the substantiating such that the review validity score is configured to represent whether the healthcare review is objectively trustworthy, whether the therapy in the healthcare review is in accord with a standard practice of the therapy, or whether the healthcare review is based on factual information corresponding to the patient or the therapy.

In an aspect, the method also includes: where the step of ascertaining includes determining that the identity of the author is a patient who received a therapy or a person involved with providing care to the patient; and where the step of transmitting includes ascertaining that the review validity score of the healthcare review satisfies a threshold configuration for adding to the staff experience record.

In an aspect, the method also includes: where the step of ascertaining includes determining that the identity of the author is another healthcare professional representing an individual or an institute other than a healthcare professional providing therapy to the patient; and where the step of transmitting includes ascertaining that the review validity score of the healthcare review satisfies a threshold configuration for adding to the staff experience record.

In accordance with aspects of the present disclosure, a system includes a memory, one or more processors in communication with the memory, and program instructions executable by the one or more processors via the memory configured to: obtain from an author, a request to post a healthcare review to a staff experience record; analyze the healthcare review to identify a subject of the healthcare review; ascertain an identity of the author as to how the author is related with the subject; validate the healthcare review based on the identity of the author, at least one a personal health record or electronic medical record corresponding to the author or the subject, and the staff experience record; determine a review validity score of the healthcare review based on results from the validating of the healthcare review; and transmit the healthcare review and the validity score to the staff experience record.

In accordance with aspects of the present disclosure, a computer program product including data representing program instructions executable by one or more processors via a memory configured to: obtain from an author, a request to post a healthcare review to a staff experience record; analyze the healthcare review to thereby identify a subject of the healthcare review; ascertain an identity of the author as to how the author is related with the subject; validate the healthcare review regarding factual basis and objectivity of content based on the identity of the author, at least one of personal health records and electronic medical records corresponding to the author or the subject, and the staff experience record; determine a review validity score of the healthcare review based on results from the validating of the healthcare review; and transmit the healthcare review and the validity score to the staff experience record.

It will be appreciated by those skilled in the art that two or more of the above-mentioned embodiments, implementations, and/or optional aspects of the present disclosure may be combined in any way deemed useful. Modifications and variations of any system and/or any computer readable medium, which correspond to the described modifications and variations of a corresponding computer-implemented method, can be carried out by a person skilled in the art on the basis of the present description.

### BRIEF DESCRIPTION OF THE DRAWINGS

These and other aspects of the present disclosure will be apparent from and elucidated further with reference to the embodiments described by way of example in the following description and with reference to the accompanying drawings, in which:

FIG. 1 shows functional components of a healthcare review validation system;

FIG. 2 shows a flowchart of a computer-implemented method performed by the healthcare review validation system of FIG. 1;

FIG. 3 shows a detailed example of how to analyze the therapy at issue by use of cause-effect modelling in block 240 of FIG. 2; and

FIG. 4 shows a computer-readable medium including data.

It should be noted that the figures are purely diagrammatic and not drawn to scale. In the figures, elements which correspond to elements already described may have the same reference numerals.

### DETAILED DESCRIPTION

The present disclosure is directed to systems and methods that validate healthcare reviews on medical therapies based on identifying patient authors and on examining data on the medical therapies in relation with the patient authors and interactions with related healthcare professionals. The systems and methods utilize other cause factors that might have affected the therapies on the patient authors, including but not limited to, therapy adherence, known therapy causation models, based health condition and lifestyle of the patient authors, and cognitive analytics tools. The present disclosure also provides techniques to validate reviews on healthcare professionals and healthcare entities based on facts presented in staff experience records and electronic medical records and other healthcare records. The systems and methods of the present disclosure improve reliability and trustworthiness of the healthcare reviews.

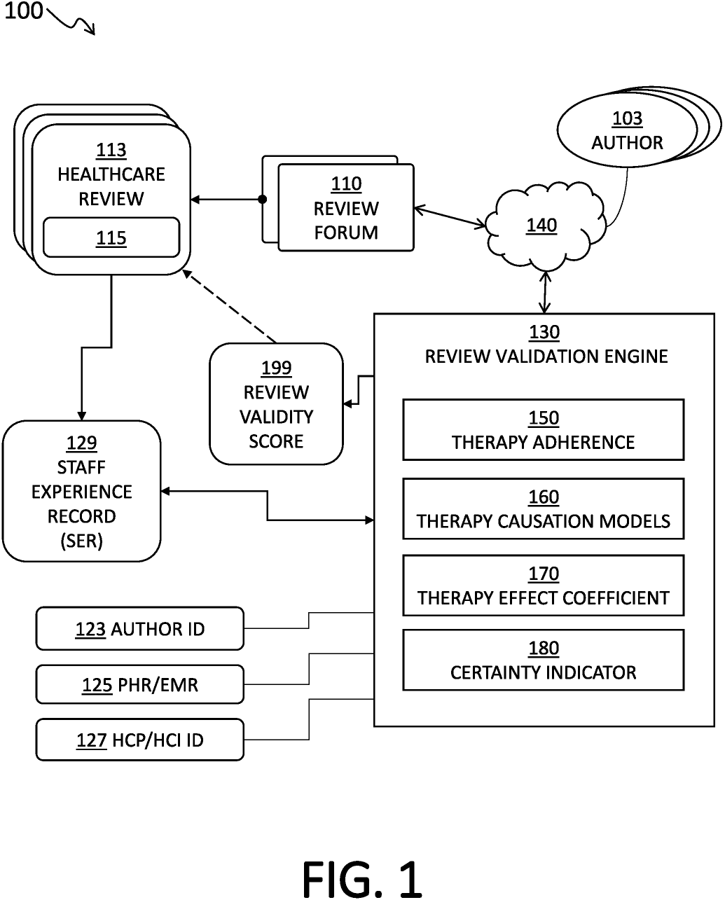

FIG. 1 shows functional components of a healthcare review validation system 100.

Conventional screening for online and other electronic reviews is often based on text analysis by natural language processing (NLP) such as word n-grams, part-of-speech n-grams, and other lexical attributes. Similar content and style in reviews from different reviewers can be suspected as a single reviewer or a web bot posting reviews assuming multiple user identities. Semantic inconsistency in numbers, pronouns, and stories in reviews can be an indication of untruthful review. Also conventionally, online and other electronic reviews can be identified with known perpetrators of false reviews spotted by odd time window of posting, frequency of posting, as well as identified by Internet Protocol (IP) addresses and/or Medium Access Control (MAC) addresses, locations, or registration identities.

The healthcare review validation system 100 may include a review forum 110 including, but not limited to, social media networks and messengers in which unsolicited reviews can be posted by the public. The review forum 110 can also be subject-oriented regarding healthcare including, but not limited to, healthcare products and services, therapies, healthcare professionals, and healthcare entities. The review forum 110 may be configured to subscribe to a review validation service to screen postings therein. In the present disclosure, the review forum 110 can include an online interface though which a plurality of healthcare reviews is attached to staff experience records 129.

An author 103 may request to add a healthcare review 113 to the staff experience record (SER) 129 via an interface of the review forum 110 via a digital communication network 140. The author 103 may be registered with the review forum 110 for access. A subject 115 of the healthcare review 113 can be a therapy, treatments, prescriptions and medicinal products, a healthcare professional, and a healthcare entity, patient experience with specific healthcare providers provided either by the patient themselves or other caregivers or others like family members close to the patient, a general recommendation of healthcare providers by patients or other caregivers or others like family members close to the patient, assessment on medical practice and peer healthcare providers by other healthcare providers, or combinations thereof. It should be noted that the author of a healthcare review 113 may be the patient, or the author may be also someone else other than the patient. For instance, a family member, a caregiver, a nurse, etc. can add a healthcare review 113. It should also be noted that the author may be a person involved with providing care to a patient, which may include providing and/or helping with therapy to a patient, or only monitoring the patient. Of course, the healthcare review will be about the patient and the care received by the patient, but it will be from another person’s perspective when the author is someone other than the patient. The validation steps disclosed herein will apply both in the case of the author being the patient or in the case of the author being someone other than the patient. The main objective is to evaluate and validate that the healthcare received as described in the review matches to the “objective” facts that can be extracted or estimated from health records (electronic medical records or personal health records), and staff experiences records.

The healthcare review validation system 100 may include a review validation engine 130 operatively coupled to the review forum 110 via the digital communication network 140. The review validation engine 130 may access health information for patients including personal health records (PHR) and electronic medical records (EMR) 125 and career information of healthcare professionals including staff experiences records (SER) 129, either directly or indirectly across the digital communication network 140. The review validation engine 130 may discover an identity of the author 103, either a patient or other caregivers or others like family members close to the patient, or a healthcare professional, including individuals and institutions.

In the PHR/EMR 125, a personal health record (PHR) is a health record where health data and other information related to healthcare of a patient is maintained by the patient. In contrast, an electronic medical record (EMR) of the PHR/EMR 125 is health information of the same patient as operated by healthcare professionals/providers (HCPs) or healthcare institutes (HCIs), such as doctors, clinics, and hospitals and contains treatment/billing data entered by clinicians to support insurance claims. The PHR/EMR 125 is to provide a complete and accurate summary of medical history of an individual which with due authorisation is accessible online, for use by both patients and HCPs from all sources. The PHR/EMR 125 can include, but are not limited to, diagnoses, treatments, prescriptions, patient-reported outcome data, lab results, and data from health monitoring devices such as wireless electronic weighing scales, fitness trackers, blood pressure monitors, and thermometers, or any other personal wellness monitoring devices. In this disclosure, the PHR/EMR 125 indicates comprehensive medical and health information on a specific patient as well as base health condition of the patient including age, body mass index, gender, lifestyle including activity level, sleep, and nutrition, regardless of the source of information, which is used by the review validation engine 130 as inputs.

The review validation engine 130 can obtain an identity of the author 103, denoted as an author ID 123 from the review forum 110, as consented by the author 103 upon posting the healthcare review 113 on the review forum 110 or within the bounds of related laws and regulations and/or for public policy regarding information transparency and data privacy.

The review validation engine 130 may also identify any healthcare professionals and healthcare institutes related with the healthcare review 113, denoted as an HCP/HCI ID 127, which can also be the author 103 or the subject 115 of the healthcare review 113, by examining data available from the PHR/EMR 125 and the SER 129.

The review validation engine 130 may assess a therapy adherence 150, indicating a numeric assessment on how well the author 103 had participated in the therapy in attending therapy sessions and in following procedural requirements, by use of the PHR/EMR 125 corresponding to the author ID 123, for the author 103 who is a patient, and content of the healthcare review 113. The review validation engine 130 may utilize known therapy causation models 160 to assess a therapy effect coefficient 170 and a certainty indicator 180 for the therapy effect coefficient 170 for the therapy addressed in the healthcare review 113. The therapy causation models 160 addresses various causes that influence the effect of the therapy at issue in the healthcare review 113, with regard to base health conditions and behaviors that affect the therapy for a patient that can be substantiated by the PHR/EMR 125. The therapy causation models 160 include, but are not limited to, an established statistical model interrelating certain causes and effects regarding the therapy at issue as well as respective probabilities in each pair from the causes and the effects.

The review validation engine 130 includes natural language processing (NLP) and other cognitive analytics (CA) mechanisms in artificial intelligence (AI) technology to have natural language text content of the healthcare review 113 analyzed and labeled for further processing in determination of the therapy adherence 150, analysis with the therapy causation model 160, determination of the therapy effect coefficient 170 and the certainty indicator 180.

The SER 129 is a body of authenticated records collating career relevant experiences of a healthcare professional. The SER 129 is authenticated records distributed across many healthcare entities medical education entities that respectively keeps their records regulated and monitored. The SER 129 of respective healthcare professionals corresponding to the HCP/HCI ID 127 includes, but are not limited to, a curriculum vitae including collated education, training, and employments, and clinical experiences, contents of each of the clinical experiences. The clinical experiences in the SER 129 respective to each HCP can include relevant parties such as patients and healthcare entities, diagnoses, treatments, prescriptions, interactions and corresponding levels of satisfaction, and status records on how each patient had abided by therapies and regimens described in each instance of the clinical experiences, health conditions regarding lifestyle and adherence to the therapies by each patient, as well as prognosis of each patient.

The SER 129 can be a part of a distributed healthcare data ecosystem. Each of the SER 129 is identified with the HCP/HCI ID 127. The EMR 125 can also be a part of the same distributed healthcare data ecosystem as the SER 129, corresponding to each patient as prepared by healthcare institutes and/or HCPs, also identified by the HCP/HCI ID 127. The SER 129 can be maintained by respective healthcare professionals associated with the HCP/HCI ID 127.

The SER 129 is distinctive from staff records kept at a human resource (HR) department at healthcare providers, as each individual healthcare professional can manage his or her own SER by adding authenticated data to provide a complete and accurate summary of each of the healthcare professional on instances of education, training, employment, and/or experiences which can be accessible online by patients and by healthcare institutes. The SER 129 can further include performance ratings, patient/treatment health outcomes, patient/peer reviews, or any other relevant performance information. Respective instances of data on the SER 129 include data reported by many sources, including but not limited to, the HCP who owns the SER 129, by other healthcare professionals, by administrators of healthcare institutes which hired the owner of the SER 129, or by patient reports or reports of other caregivers of the patient or others like family members close to the patient.

The review validation engine 130 may determine a review validity score 199 based on the factors of the therapy adherence 150, the therapy causation models 160, the therapy effect coefficient 170, and the certainty indicator 180. The review validity score 199 may exclude any personal information of the author 103 and any healthcare professionals relevant to the subject 115 of the healthcare review 113, as governed by data protection laws and regulations. Components of the healthcare review validation system 100 can run on respective servers, cloud computing nodes, or any other computing platforms that can be geographically distributed but operatively coupled via the digital communication network 140 and other communication interface.

The review forum 110 may receive the review validity score 199 and meditate presentation of the healthcare review 113 based on a predefined moderation rule in accordance with the magnitude of the review validity score 199.

The healthcare reviews 113 are to be more trustworthy and fact-based than other online or other electronic postings particularly because safety and health of the public can be greatly affected by such healthcare reviews 113. Also, because certain healthcare entities and HCPs can be benefited or harmed by certain reviews, the healthcare reviews 113 are to be objective and fair on the effects and performances of such healthcare entities and HCPs. From the perspective of public knowledgebase, the healthcare reviews 113 on therapies, medications, treatments, and/or HPCs and healthcare entities, that is, doctors and hospitals, are to be presented as comprehensively as possible, particularly because results for individual patients can differ based on various context of the therapy at issue. Further, certain patients would post the healthcare review 113 that inadvertently reveals sensitive information of themselves protected under health information privacy laws and regulations such as the Health Insurance Portability and Accountability Act (HIPAA), which can be protected by the review forum 110 with content screening. The healthcare review validation system 100 presented herein is motivate by improving integrity in health information provided in the healthcare review 113 and protecting privacy and interests of the public, healthcare entities and HCPs, as well as patients.

The healthcare review validation system 100 can be applied in the SER 129 of a distributed healthcare data ecosystem, the review forum 110 for publishing the healthcare review 113, any medical therapy knowledge management systems, and/or any medical applications.

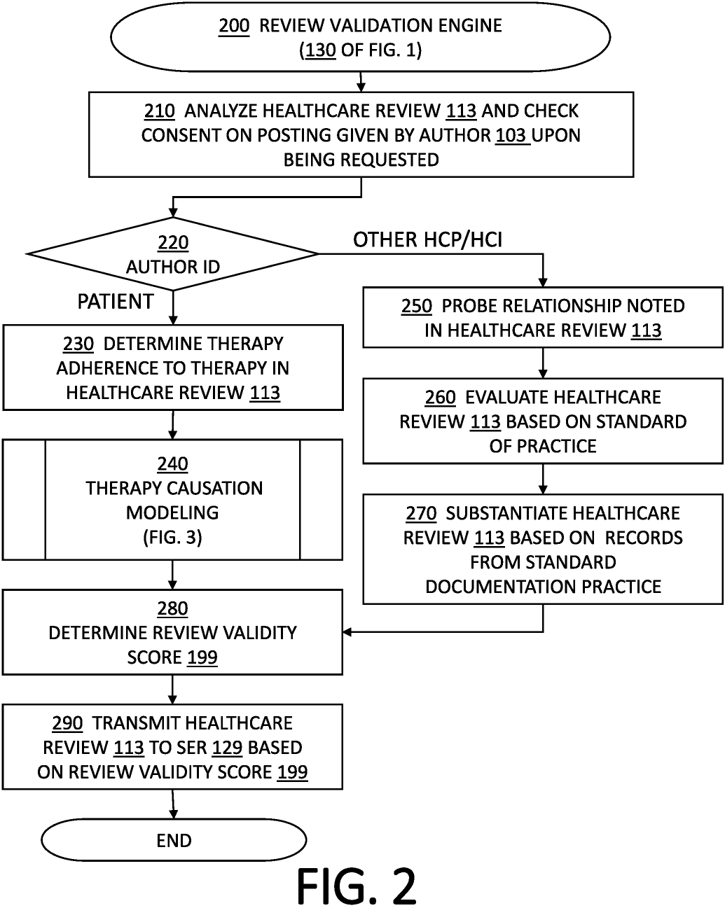

FIG. 2 shows a flowchart 200 of a computer-implemented method performed by the healthcare review validation system 100 of FIG. 1.

The review validation engine 130 of the healthcare review validation system 100 performs blocks 230 through 260 on the healthcare review 113 on healthcare products and services as experienced by the author 103 who is a patient or other caregivers of the patient or others like family members close to the patient. The review validation engine 130 performs blocks 270 through 290 and blocks 250 and 260 on the healthcare review 113 posted by healthcare professionals. As noted above, the PHR/EMR 125 on patient healthcare record and data and the SER 129 on HCP career record are respectively available from distributed healthcare data ecosystems, which is accessed by the review validation engine 130 in order to validate the healthcare review 113 based on factual basis substantiated by the data from the PHR/EMR 125 and the SER 129. The PHR/EMR 125 and the SER 129 are representative of all trustworthy data sources to check the healthcare review 113 against by the review validation engine 130.

Factual data that are utilized to substantiate the healthcare review 113 by the review validation engine 130 include but are not limited to, medical equipment data and computer application data as used in treatment and monitoring of the patient, sale statistics in volumes and market share for a particular medical product at issue in the healthcare review 113, relationships amongst HCPs who authors the healthcare review 113 and who is being discussed in the healthcare review 113 based on the SER 129 for relationships throughout the respective careers as well as employments, educations, trainings, and other relationships between HCPs and healthcare institutes. For example, if the author 103 is a healthcare professional posting the healthcare review 113 on an affiliate healthcare entity or on a competitor healthcare institute, at least such relationship should be revealed to provide better understanding of the healthcare review 113.

In block 210, the review validation engine 130 obtains a request to post the healthcare review 113 onto the review forum 110 and analyzes content of the healthcare review 113. The review validation engine 130 checks if the author 103 logged in on the review forum 110 with a legitimate account and checks if the author 103 consented to validation of the healthcare review 113 for posting with the review forum 110. Then, review validation engine 130 proceeds with block 220.

The review validation engine 130 can be a separate application or an infrastructure providing a subscription-based review validation service for the review forum 110 of the healthcare review validation system 100. The review validation engine 130 may be operatively coupled to conventional NLP tools and review screening mechanisms based on text analysis for syntax and semantics, as noted above. However if reviews are based upon star ratings or other fixed format Question/Answer approaches such NP tools may not be required. The review validation engine 130 have the content of the healthcare review 113 analyzed and identifies the subject 115 of the healthcare review 113 including, but not limited to, a therapy, a healthcare professional, and a healthcare entity, or combinations thereof. The healthcare review 113 regarding medical products and medications can be easily checked by use of product documentations and market statistics, so the review validation engine 130 focused on the subject 115 of the healthcare review 113 regarding treatments and interactions between a patient and HCPs/HCIs, which are more likely to be misunderstood and/or misrepresented than product descriptions and market statistics.

In block 220, the review validation engine 130 determines if the author 103 is a patient or other caregivers or others like family members close to the patient or other healthcare professional or healthcare institutes (HCP/HCI). If the review validation engine 130 determines that the author 103 is a patient, then the review validation engine 130 proceeds with block 230. If the review validation engine 130 determines that the author is other HCP/HCI, then the review validation engine 130 proceeds with block 270.

The review validation engine 130 determines the author ID 123 based on registration information and/or user profile sent from the review forum 110. The author ID 123 can be set either as a patient identity or an authoring HCP/HCI identity. In contrast with the author ID 123, the HCP/HCI ID 127 indicates one or more healthcare professionals or healthcare institutes either discussed in the healthcare review 113 or have any interests or relationship that may affect the objectivity of the healthcare review 113, identified by the SER 129 as noted above.

In block 230, the review validation engine 130 determines the therapy adherence 150 to a therapy in the healthcare review 113, quantified as a probability, for example. The therapy adherence 150 in the review validation engine 130 is to establish a fact that the author 103 actually received the therapy addressed in the healthcare review 113 as a patient. Then, the review validation engine 130 proceeds with block 240.

In certain embodiments, the review validation engine 130 retrieves and examines the PHR/EMR 125 of a patient corresponding to the author 103 for any evidence of the therapy in the healthcare review 113 acclaimed as received by the author 103. To analyze the PHR/EMR 125, the review validation engine 130 utilizes the NLP tools for text classification on medical glossaries including synonyms, and a taxonomy of medical terms, similarly with the text analysis for the healthcare review 113.

The review validation engine 130 can be configured to set the therapy adherence 150 as a maximum value, for example, 1.0, if the PHR/EMR 125 proving that the patient had attended all therapy sessions held in hospitals. With available therapy attendance record in the PHR/EMR 125, the review validation engine 130 can be configured to reduce the therapy adherence 150 corresponding to the number of missing sessions of the therapy proportional to the total number of therapy sessions discussed in the healthcare review 113. If the PHR/EMR 125 of the author 103 does not include any record of the therapy in the healthcare review 113, then the review validation engine 130 will set the therapy adherence 150 to a minimum value available, for example, 0.0.

In certain embodiments, the review validation engine 130 utilizes data recorded by one or more personal wellness devices of the author 103, also available from the PHR/EMR 125 monitoring various healthcare related events in relation with the therapy in the healthcare review 113. Examples of the healthcare related events include, but not limited to, meals and nutritional information, activities and exercise events, scheduled intakes of medications, appointments related with the therapy in the healthcare review 113. Respective healthcare events are timestamped either as provided by such personal wellness devices of the author 103 or as recorded by an HCI hosting the therapy for the author 103.

In certain embodiments, the therapy in the healthcare review 113 is of a type that is self-conducted by the patient. The review validation engine 130 would identify activity plans or a protocol corresponding to the therapy commented in the healthcare review 113 and set the parameters of the therapy adherence 150. For example, if the therapy protocol includes a certain number of daily medication, exercise requirement for certain amount, intensity, and frequency, as well as a series of check-up visits with an HCP for a certain therapy period, the review validation engine 130 can set the therapy adherence 150 proportional to each item in the therapy protocol and account each occasion for the items prescribed in the therapy protocol based on the data log of the one or more personal wellness devices of the author 103. In the same example as above, the review validation engine 130 can set 0.33 for daily medication intakes, the exercise regimen, and attendance with the check-up visits, respectively, toward the therapy adherence 150 of 1.00, and add values assigned for each occasion for each item throughout a duration required for the therapy in the healthcare review 113, beginning from the therapy adherence 150 value of 0.01, such that a perfect adherence to the therapy protocol will result in the therapy adherence 150 value of 1.0.

In cases where the therapy protocol includes certain restrictions for the patient not to do, with respect to types of activities to refrain from or a list of foods not to eat, the review validation engine 130 can also configure a certain penalty for each occasion where the author 103 violated such restrictions. The review validation engine 130 checks for records of such violations against the records of the personal wellness devices of the author 103 and reduces the therapy adherence 150 by the preconfigured penalty for each occurrence of the violations.

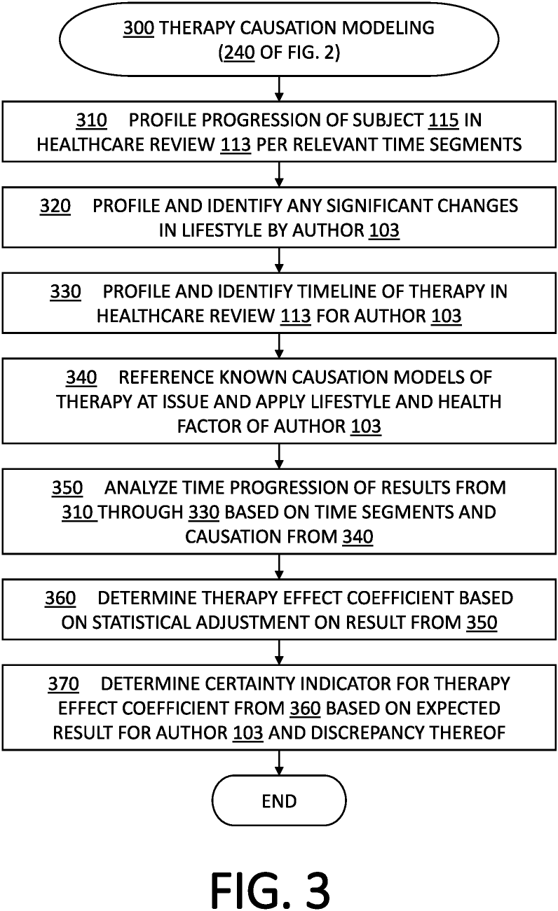

In block 240, the review validation engine 130 analyzes how effective the therapy can be based on the therapy causation models 160 for the author 103 based on circumstances of the therapy in the healthcare review 113, provided that the author 103 has abided by the therapy protocol as shown in the therapy adherence 230 of block 230. Detailed operations of the therapy effect analysis are presented in FIG. 3 and corresponding description. Then, the review validation engine 130 proceeds with block 280.

The purpose of therapy causation model analysis is to ascertain how effective the therapy could be based on the therapy causation models 160 under the circumstances unique for the author 103, as reflected in the therapy effect coefficient 170, such that the review validation engine 130 can objectively determine if the healthcare review 113 is consistent with a realistic effect of the therapy that the author 103 could expect under the circumstances, as a result of the base health conditions of the author 103. Because the healthcare review 113 would be more informative and beneficial to the public if the contexts of the therapy on the author 103 are presented with the healthcare review 113 than without the contexts, at least for factors identified as affecting the effect of the therapy in the therapy causation model 160. Further, the review validation engine 130 can profile certain timed events of the author 103 within a period that can affect the therapy in the healthcare review 113 as well as the time of posting the healthcare review 113 in relation with the completion of the therapy and a time period in which any effect of the therapy can be expected according to the therapy causation models 160. The review validation engine 130 also determines how reliable the analysis of the therapy causation models 160 on the contextual factors specific to the author 103, referred to as a certainty indicator 180.

The therapy causation models 160 are selected from medical statistics databases and medical knowledgebases operatively coupled to the review validation engine 130 for data interface. Because contextual factors specific to the author 103 regarding the therapy in the healthcare review 113 can alter the effectiveness of the therapy for the author 103 as a patient, the review validation engine 130 determines how effective the therapy in the healthcare review 113 is supposed to be based on such contextual factors uniquely associated with the patient. Examples of the contextual factors specific to the patient include, but not limited to, base health conditions such as including age, body mass index, gender, medical history including past diagnoses and treatments, allergies and susceptibilities including family medical history, lifestyle including activity level, sleep, and nutrition, as well as any life events that can affect the therapy in the healthcare review 113.

In block 250, the review validation engine 130 identifies the HCP/HCI who is the author 103 of the healthcare review 113 and examines a relationship between the author 103 and the HCP/HCI who is the subject 115 of the healthcare review 113. Then, the review validation engine 130 proceeds with block 260.

In certain embodiments of block 250, the review validation engine 130 searches the SER 129 with the author ID 123 identifying the HCP/HCI who is the author 103 as determined in block 220 above. The review validation engine 130 may also search, if available, the SER 129 of the HCP/HCI ID 127 identifying the HCP/HCI who is the subject 115 of the healthcare review 113, also determined from block 220 above. The review validation engine 130 may compare the respective SERs corresponding to both the author ID 123 and if available the HCP/HCI ID 127 for any overlap in training, education, employment, as well as geographical locations to determine if the respective healthcare professionals corresponding to the author ID 123 and the HCP/HCI ID 127 are personally acquainted or related in any way, to check if there can be any favorable or unfavorable relations to bias the healthcare review 113. Examples of such relations include, but are not limited to, shared alma maters, shared previous or current employments, candidacy for a same position, colleagues, co-authors, teacher-student relationship, accreditations and professional association memberships, or any other professional relationships appearing on the SER 129.

The review validation engine 130 can further extract any particular interests of the respective healthcare professionals corresponding to the author ID 123 and the HCP/HCI ID 127 to check for any relationships not directly appearing on the SER 129 — or indeed if the HCP/HCI ID 127 does not have a HCP — but can bias the healthcare review 113, such as being employed at competitor HCIs, any business relationship in the respective institutes for the respective healthcare professionals corresponding to the author ID 123 and the HCP/HCI ID 127. The review validation engine 130 accesses to healthcare market data in order to determine if the respective healthcare professionals corresponding to the author ID 123 and the HCP/HCI ID 127 have any business interests aligned or conflicted. The healthcare market data can be used to preprocess the SER 129 for tagging HCIs with such attributes of relationships amongst HCIs, particularly for competitions and affiliations.

In addition to any bias, the review validation engine 130 can check the SERs of the respective healthcare professionals corresponding to the author ID 123 and the HCP/HCI ID 127 to see if the author represented by the author ID 123 is qualified to assess professional performance of the HCP/HCI of the subject 115, for example, at least having a certain level of medical training, a certain number of years of experience either in generic medicine or in the same area as the subject 115, depending on the complexity and the content of the healthcare review 113.

In block 260, the review validation engine 130 evaluates the healthcare review 113 based on a standard of practice corresponding to the area of medicine and for a particular case that appears in the healthcare review 113. The review validation engine 130 determines if the healthcare review 113 is objective and consistent with the standard of practice related. Then, the review validation engine 130 proceeds with block 270.

The standard of practice regarding the area of medicine and cases with contextual factors to evaluate the healthcare review 113 can be referred from the medical statistics databases and the medical knowledgebases as used in block 240 for the therapy causation models 160. The review validation engine 130 checks if the healthcare review 113 calls upon any non-standard practices to criticize or to compliment the HCP/HCI of the subject 115 identified by the HCP/HCI ID 127.

In block 270, the review validation engine 130 substantiates content of the healthcare review 113 based on records from standard documentation practice, including medical equipment use logs and local and remote computer application logs and other institutional records by the HCI as a venue of the therapy of the healthcare review 113, the PHR/EMR 125 of similar cases and therapies. The review validation engine 130 determines if the healthcare review 113 is proven as being factually grounded or based on factual information, particularly in cases where any specific assertion of non-standard practice that could be unfavorable to the HCP/HCI of the subject 115 identified by the HCP/HCI ID 127. Then, the review validation engine 130 proceeds with block 280.

In block 280, the review validation engine 130 determines the review validity score 199 of the healthcare review 113 depending on the author 103. If the author 103 is a patient, the review validation engine 130 determines the review validity score 199 based on a combination of the therapy adherence 150 resulting from block 230, the therapy effect coefficient 170, and the certainty indicator 180 corresponding to the therapy effect coefficient 170 resulting from block 240 if the author 103 is a patient. If the author 103 is other HCP/HCI, the review validation engine 130 determines the review validity score 199 as a combination of results from blocks 250 through 260. Regardless of the identity of the author 103, the review validity score 199 is to indicate whether the healthcare review 113 is unbiased and substantiated based on all records available to the review validation engine 130, including but not limited to the PHR/EMR 125, the SER 129, and other medical knowledgebase and statistics. Then, the review validation engine 130 proceeds with block 290.

In block 290, the review validation engine 130 transmits the healthcare review 113 to the SER 129 based on the review validity score 199. The review validation engine 130 can mediate the healthcare review 113 based on the review validity score 199 resulting from block 280 for objectivity and validity, or to protect privacy of the author 103 as well as the HCP/HCI at issue in the subject 115 of the healthcare review 113 within the bounds of applicable law. Then, the review validation engine 130 terminates processing for the healthcare review 113.

In certain embodiments, the review validation engine 130 can report the healthcare review 113 as mediated by the threshold posting configurations per the review validity score 199 to the SER 129. The review validation engine 130 can also apply the therapy adherence 150, the therapy effect coefficient 170, and the certainty indicator 180 to the healthcare review 113 along with the review validity score 199 for mediating the healthcare review 113.

In certain embodiments, the author 103 would be requested to give consent to the validation and mediation of the healthcare review 113 and the threshold configurations per the review validity score 199 as performed by the review validation engine 130 for adding to the SER 129 or optionally posting on the review forum 110. In cases where the author 103 had not given consent to the validation and mediation, the review forum 110 can optionally post the healthcare review 113 without the review validity score 199 with marking such as “unknown review validity” or “review not validated”, or not to publish the healthcare review 113, depending on publication policy of the review forum 110 or the threshold configurations per the review validity score 199.

The healthcare review 113 would be screened for any identifiable names of individuals or other sensitive information as doctor-patient relationship is privileged and should be kept confidential. Thus, if the author 103 inadvertently disclosed any names of HCP/HCI that can be identified and related with the author 103, including the name of the author 103, the review validation engine 130 would mask or change into fictional names in the healthcare review 113 or mark such names to be masked or changed prior to adding the healthcare review 113 with the SER 129 and optionally to the review forum 110. Particularly, HCPs and HCIs are responsible to keep the confidentiality regarding identity of patients of the HCPs and HCIs and content of medical information for the patients as noted earlier. The review forum 110 can keep the privacy of the patients and confidentiality of medical information by masking any identifiable names or changing such names into fictional names with notice of such changes in names.

In certain embodiments, the review validation engine 130 can apply a predefined threshold on the review validity score 199 as configured by the review forum 110 such that the review forum 110 will post the healthcare review 113 that meets the healthcare review 113 only when the review validity score 199 corresponding to the healthcare review 113 meets the predefined threshold on the review validity score 199. By applying the predefined threshold on the review validity score 199 for the healthcare review 113, trustworthiness and objectivity of the healthcare reviews 113 of the SER 129 will be assured.

In certain embodiments of block 290, the the review validation engine 130 can also rate the healthcare review 113 based on the review validity score 199 of the healthcare review 113, by use of a scale with a predefined number of ratings, and add to the SER 120 based on the rating associated with the healthcare review 113.

In certain embodiments of the healthcare review validation system 100, the SER 129 can be updated with the healthcare review 113 by patients as associated with the review validity score 199. The healthcare review 113 in the SER 129 would include sensitive information such as patient identity unlike the healthcare review 113 published in the review forum 110 as the SER 129 is kept confidential and complete information in the healthcare review 113 is more valuable to the HCP/HCI who owns the SER 129.

In certain embodiments of the healthcare review validation system 100, the SER 129 of the HCP/HCI who is of the subject 115 can be updated with the healthcare review 113 by other HCPs/HCIs, as peer review of performances of an owner of the SER 129.

In certain embodiments of the healthcare review validation system 100, the subject 115 of the healthcare review 113 that discusses performances of the HCP identified by the HCP/HCI ID 127 can be first checked with the SER 129 owned by the HCP of the subject 115. If the SER 129 of the HCP of the subject 115 is consistent with content the healthcare review 113 regarding diagnoses, treatments, and interactions, then the review validation engine 130 can validate the healthcare review 113 without examining the PHR/EMR 125. If the SER 129 of the HCP of the subject 115 is not consistent with content the healthcare review 113 on the diagnoses, the treatments, and the interactions, then the review validation engine 130 would further examine the PHR/EMR 125 to determine any causes of such inconsistencies external to the diagnoses, the treatments, and the interactions such as certain intervening actions of other HCPs, other hospital staffs or assistants, of patients themselves or even performance of hospital equipment and medical devices including hospital infrastructure and/or utility system of hospitals.

In certain embodiments of the healthcare review validation system 100, the review validation engine 130 would invite other HCP/HCI that is not related with the subject 115 of the healthcare review 113 for objective peer review on performances of the HCP/HCI of the subject 115, regarding factors such as the complexity of the case discussed in the healthcare review 113, an average outcome from departments and/or hospitals for cases similar to the case discussed in the healthcare review 113, detailed comparison with standard treatment protocol and/or result for similar cases as the case discussed in the healthcare review 113.

In certain embodiments of the healthcare review validation system 100, the review validation engine 130 can utilize medical equipment use logs and other hospital system data (e.g., from local or remote computer applications) other than the PHR/EMR 125 specific to the patient corresponding to the author ID 123 to validate the healthcare review 113, particularly when the area of medicine is primarily practiced with a use of medical equipment such as radiology, other medical imaging and therapies using computerized systems. Such medical equipment use logs can also substantiate the healthcare review 113 such that the healthcare review 113 can be used to update the SER 129 for the HCP/HCI of the subject 115 whose performance is discussed in the healthcare review 113. Similarly, use logs from any other medical equipment that can identify a patient and/or responsible HCP can be utilized to update the SER 129 for the HCP/HCI of the subject 115. Examples of medical equipment that can identify the patient and/or responsible HCP include, but not limited to, medication dispensers, patient monitoring devices for vital signs, electrocardiogram, or blood oxygen levels. Use data from certain medical equipment that can only be activated with a radio frequency identification (RFID) of an HCP can also be used to substantiate the healthcare review 113 regarding performances of the HCP as well as to update the SER 129 for the HCP as a reliable source of information.

In certain embodiments of the healthcare review validation system 100, the review validation engine 130 can be retrieved from medical equipment, encoded, and stored in the SER 129 of the HCP/HCIs who generated the logs or otherwise related to the logs. Other data generated by hospital infrastructure that are not included in the EMR 125 such as use record for printers including information such as an HCP who ordered to print a file, the file that had been printed, location of a printer received the order and location of a system from which an order to print was sent, and the time of the printing, can also be identified and stored in the healthcare review validation system 100 to assess the review validity score 199 of the healthcare review 113.

FIG. 3 shows a detailed example of how to analyze the therapy at issue by use of known therapy causation models in block 240 of FIG. 2.

In block 310, the review validation engine 130 profiles the progression of the subject 115 in the healthcare review 113 per relevant time segments for determining the therapy effect coefficient 170 and the certainty indicator 180 per time segment. Then, the review validation engine 130 proceeds with block 320.

In certain embodiments, the review validation engine 130 checks the healthcare review 113 for any description of time that can identify when the therapy of the subject 115 had occurred.

In block 320, the review validation engine 130 profiles and identifies any significant changes in behavior, lifestyle, environment, or context by the author 103. Then, the review validation engine 130 proceeds with block 330.

In certain embodiments, the review validation engine 130 examines the PHR/EMR 125 to profile timed events regarding any significant changes in the lifestyle, behaviors, environment, or context of the author 103 that may affect effectiveness of the therapy in relation with the time progression identified in the subject 115 from block 310 above. For example, the online therapy causation model 160 can identify that the author 103 started or stopped smoking, that the author 103 gained or lost an excessive amount of weight, that the author 103 suffered any bodily injury unrelated with the therapy, that the author 103 had been hospitalized or diagnosed with another condition that did not appear in the subject 115 of the healthcare review 113.

In block 330, the review validation engine 130 profiles and identifies the timeline of the therapy in the healthcare review 113 for the author 103, based on the PHR/EMR 125 of the author 103. Then, the review validation engine 130 proceeds with block 340.

In block 340, the review validation engine 130 searches any therapy causation model relevant to the subject 115 of the healthcare review 113 for time segments regarding effect of the therapy and applies the behaviors, lifestyle, environment, and/or context changes of author 103 as profiled with time in block 330. Then, the review validation engine 130 proceeds with block 350.

In certain embodiments, the review validation engine 130 sets the time segments in accordance with timeframes of the therapy causation models 160 applicable for the therapy in the subject 115. For example, a therapy causation model for diagnosis A amongst the therapy causation models 160 would specify the effect of the therapy would be checked at 3 months, 6 months, and 1 year after the completion of the therapy, and a full effect of the therapy can be known only after a couple of years after the completion of the therapy. Accordingly, the review validation engine 130 sets the time segments for the progression of the subject 115 regarding a therapy for diagnosis A with five time segments including a first period between the completion of the therapy and 3 months thereafter, a second period between 3 months and 6 months after the completion of the therapy, a third period between 6 months and 12 months after the completion of the therapy, a fourth period between 1 year and 2 years, and a fifth period after the second anniversary of the completion of the therapy.

In the same example, the therapy causation model for diagnosis A would also specify health factors such as the age and any basic health metrics, the physical fitness, any chronic conditions, and other diagnoses for author 103 to determine how effective the therapy in the subject 115 can be for the author 103.

In block 350, the review validation engine 130 analyzes the time progression of the subject 115 profiled from block 310, healthcare related changes and issues of the author 103 profiled from block 320, and the timeline of the therapy determined from block 330, in comparison with time segments and causations the therapy causation model 160 as identified in block 340. Then, the review validation engine 130 proceeds with block 360.

In certain embodiments, the review validation engine 130 ascertains if any changes in the progression of the subject 115 of the healthcare review 113 coincides with a beginning of the therapy of the healthcare review 113. In certain embodiments, the review validation engine 130 will also ascertains if any changes in the progression of the subject 115 of the healthcare review 113 coincides with any significant change in lifestyle or health-related behaviors of the author 103 around or after a time when such change would affect the subject 115 of the healthcare review 113. In certain embodiments, the review validation engine 130 will ascertain if any changes in the progression of the subject 115 of the healthcare review 113 coincides with a start or any significant change in adherence to other therapies around or after a time when such change would affect the subject 115 of the healthcare review 113.

In block 360, the review validation engine 130 determines the therapy effect coefficient 170 that indicates to what extent the progression of the health issues in the healthcare review 113 would be the effect of the therapy named by the author 103 in the healthcare review 113, that is, to what extent content of the healthcare review 113 would have been caused by the therapy in the healthcare review 113. Accordingly, the therapy effect coefficient 170 also reversely accounts effects of lifestyle, behavioral, environment, or context factors other than the therapy on the healthcare issues of the subject 115, as profiled and identified in block 320. The review validation engine 130 determines the therapy effect coefficient 170 by statistically adjusting the result from block 350. Then, the review validation engine 130 proceeds with block 370.

By using known statistical methods, the review validation engine 130 calculates any explainable effects on the subject 115 of the healthcare review 113 of time-sensitive causes profiled in blocks 310 through 340, including but not limited to, the therapy on the author 103, any significant changes in lifestyle, health behavior, environment, or context of the author 103, and the beginning of another therapy or any significant changes in adherences to other ongoing therapies.

For the same example as in the healthcare review 113 for the therapy on the diagnosis A as above, the review validation engine 130 can increase the therapy effect coefficient 170 beginning the third period if the author 103 stopped smoking 6 months after the completion of the therapy, which would statistically enhance the effect of the therapy as noted in the therapy causation model 160. On the other hand, the review validation engine 130 can decrease the therapy effect coefficient 170 beginning the second period if the author 103 started smoking 4 months after the completion of the therapy, which would statistically diminish the effect of the therapy according to the therapy causation model 160.

In block 370, the review validation engine 130 determines the certainty indicator 180 for the therapy effect coefficient 170 resulting from block 360 based on an expected result for author 103 and discrepancy thereof, as analyzed in block 350. Then, the review validation engine 130 terminates operations in FIG. 3 and proceeds with block 250.

In the same example as above, the review validation engine 130 can set the value of the certainty indicator 180 to be 0.95, for example, for a certain value of the therapy effect coefficient 170 after 2 years from the completion of the therapy regarding improvement of the symptoms of diagnosis A according to the therapy causation model 160, indicating that the therapy effect coefficient 170 is 95 % likely to be accurate.

In certain embodiments, for the same example as the healthcare review 113 on the therapy of diagnosis A with therapy causation model time segments as above, the review validation engine 130 checks in which time segment the healthcare review 115 has been written to determine if any health issue discussed in the healthcare review 113 is an effect of the therapy addressed in the healthcare review 113. The therapy effect coefficient 170 would indicate how much of the health issue in the healthcare review 113 would have been affected by the therapy in the subject 115, and the certainty indicator 180 would indicate how statistically reliable the therapy effect coefficient 170 can be based on available data including, but not limited to, the PHR/EMR 125 and the SER 129, based on the therapy causation models 160.

In the same example, if the review validation engine 130 discovers that the healthcare review 113 was attributing improvement in symptoms of diagnosis A at 1 month after the completion of the therapy, the therapy effect coefficient 170 and the certainty indicator 180 would be very low, as the therapy causation model 160 indicates that the therapy would be fully effective after 2 years from the completion of the therapy. Conversely, if the review validation engine 130 discovers that the healthcare review 113 was attributing improvement in symptoms of diagnosis A 2 years after the completion of the therapy, the therapy effect coefficient 170 and the certainty indicator 180 would be significantly higher than the therapy effect coefficient 170 and the certainty indicator 180 instances at 1 month after the therapy as above, depending on a selected statistical assessment method.

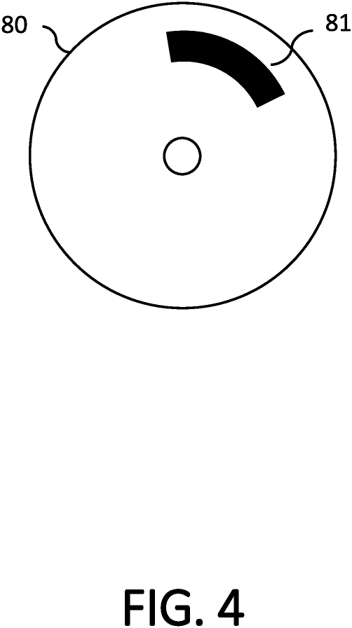

FIG. 4 shows an optical disc 80 as an example of a computer-readable medium comprising data.

The methods operative in the healthcare review validation system 100 may be implemented on a computer as a computer implemented method, as dedicated hardware, as firmware, or as a combination thereof. As also illustrated in FIG. 4, instructions for the computer, e.g., executable code, may be stored on a computer readable medium 80, e.g., in the form of a series 81 of machine-readable physical marks and/or as a series of elements having different electrical, e.g., magnetic, or optical properties or values. The medium 80 may be transitory or non-transitory. Examples of computer readable mediums include memory devices, optical storage devices, integrated circuits, servers, online software, etc.

Examples, embodiments or optional features, whether indicated as non-limiting or not, are not to be understood as limiting the present disclosure as claimed.

While the present disclosure has been illustrated and described in detail in the drawings and foregoing description, such illustration and description are to be considered illustrative or exemplary and not restrictive; the present disclosure is not limited to the disclosed embodiments.

For example, it is possible to operate the present disclosure in an embodiment wherein a computer implemented method includes: obtaining, by one or more processors, from an author, a request to post a healthcare review in a review forum; analyzing, by the one or more processors, the healthcare review to thereby identify a subject of the healthcare review by use of natural language processing tools; ascertaining, by the one or more processors, an identity of the author as to how the author is related with a therapy in the subject; validating, by the one or more processors, the healthcare review regarding factual basis and objectivity of content in the subject based on the identity of the author, personal health records and electronic medical records corresponding to the author or the therapy, staff experience records corresponding to the author or the therapy, and available medical knowledgebase including one or more known therapy causation models; determining, by the one or more processors, a review validity score of the healthcare review indicating how objectively trustworthy and factually grounded the healthcare review is based on results from the validating; and publishing, by the one or more processors, the healthcare review on the staff experience record based on threshold configurations corresponding to the review validity score to thereby inform readers of the healthcare review how valid the healthcare review is, where the step of ascertaining including: determining that the identity of the author is a patient who received the therapy in the subject; the step of validating including: determining a therapy adherence by the patient indicating how well the patient participated in the therapy in the subject, based on personal health records and electronic medical records of the patient and staff experience records of a healthcare professional providing the therapy; determining a therapy effect coefficient indicating to what extent content of the healthcare review would have been caused by the therapy by use of therapy causation modeling based on a combination of the personal health records and electronic medical records of the patient, the staff experience records of the healthcare professional, and one or more known therapy causation models; and determining a certainty indicator corresponding to the therapy effect coefficient indicating how statistically reliable the therapy coefficient is; and the step of determining the review validity score including: combining the therapy adherence, the therapy effect coefficient, and the certainty indicator by use of a selected statistical method to indicate how valid the healthcare review by the patient is in view of all available data, or the step of ascertaining including: determining that the identity of the author is another healthcare professional other than a healthcare professional providing the therapy; the step of validating including: probing a relationship between the author and the healthcare professional, based on respective staff experience records of the author and the healthcare professional to see if the relationship is likely to bias the healthcare review positively or negatively; evaluating the healthcare review based on a standard practice regarding the therapy; and substantiating the healthcare review based on records from standard documentation practice regarding the therapy including the electronic medical records corresponding to the therapy and data logs from medical equipment and computer applications (local or remote) used in the therapy; and the step of determining the review validity score including: combining the relationship between the author and the healthcare professional and respective results from the evaluating and the substantiating such that the review validity score would represent aspects of how objective the healthcare review is, if the therapy in the healthcare review is according to the standard practice of the therapy, and how factually grounded the healthcare review is, and ascertaining that the author had consented with a validation of the healthcare review; masking any names in the healthcare review that can identify the author or a healthcare professional in the subject, by use of respective fictional names; and publishing, in the review forum, the healthcare review with the fictional names, a notification of the fictional names, and the review validity score.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user’s computer, partly on the user’s computer, as a stand-alone software package, partly on the user’s computer and partly on a remote computer or entirely on the remote computer or server. In the latter scenario, the remote computer can be connected to the user’s computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A computer implemented method comprising:
obtaining, by one or more processors, from an author, a request to post a healthcare review on a staff experience record;
analyzing, by the one or more processors, the healthcare review to identify a subject of the healthcare review;
ascertaining, by the one or more processors, an identity of the author as to how the author is related with the subject;
validating, by the one or more processors, the healthcare review based on the identity of the author, at least one of personal health records and electronic medical records corresponding to a patient or the subject, and the staff experience record;
determining, by the one or more processors, a review validity score of the healthcare review based on results from the validating of the healthcare review; and
transmitting, by the one or more processors, the healthcare review and the validity score to the staff experience record.

2. The computer implemented method of claim 1,
wherein the step of ascertaining comprises determining that the identity of the author is the patient who received a therapy or a person involved with providing care to the patient; and
wherein the step of validating further comprises determining a therapy adherence by the patient indicating how well the patient participated in the therapy based on at least one of the personal health records and the electronic medical records of the patient and one or more staff experience records of one of more healthcare professionals providing the therapy.

3. The computer implemented method of claim 1,
wherein the step of ascertaining comprises determining that the identity of the author is the patient who received a therapy or a person involved with providing care to the patient; and
wherein the step of validating further comprises determining a therapy effect coefficient indicating to what extent content of the healthcare review was caused by the therapy by use of therapy causation modeling based on at least one of the personal health records and the electronic medical records of the patient, one or more staff experience records of one or more healthcare professionals providing the therapy, and one or more known therapy causation models.

4. The computer implemented method of claim 1,
wherein the step of ascertaining comprises determining that the identity of the author is the patient who received a therapy or a person involved with providing care to the patient; and
wherein the step of validating further comprises determining a certainty indicator corresponding to a therapy effect coefficient indicating how statistically reliable a therapy coefficient is.

5. The computer implemented method of claim 1,
wherein the step of ascertaining comprises determining that the identity of the author is the patient who received a therapy or a person involved with providing care to the patient; and
wherein the step of determining the review validity score comprises combining a therapy adherence, a therapy effect coefficient, and a certainty indicator by use of a selected statistical method to indicate how valid the healthcare review by the patient is in view of all available data.

6. The computer implemented method of claim 1,
wherein the step of ascertaining comprises determining that the identity of the author is the patient who received a therapy or a person involved with providing care to the patient; and
wherein the step of validating further comprises determining a therapy effect coefficient indicating to what extent content of the healthcare review would have been caused by the therapy by use of therapy causation modeling based on at least one of the personal health records and the electronic medical records of the patient, one or more staff experience records of one or more healthcare professionals providing the therapy, and one or more known therapy causation models, wherein the therapy causation modeling comprises profiling a time progression of the subject in the healthcare review.

7. The computer implemented method of claim 1,
wherein the step of ascertaining comprises determining that the identity of the author is the patient who received a therapy or a person involved with providing care to the patient;
wherein the step of validating further comprises determining a therapy effect coefficient indicating to what extent content of the healthcare review would have been caused by the therapy by use of therapy causation modeling based on a combination of the personal health records or electronic medical records of the patient, one or more staff experience records of one or more healthcare professionals providing the therapy, and one or more known therapy causation models, wherein the therapy causation modeling comprises profiling a timeline of the therapy for the patient.

8. The computer implemented method of claim 1,
wherein the step of ascertaining comprises determining that the identity of the author is the patient who received a therapy or a person involved with providing care to the patient;
wherein the step of validating further comprises determining a therapy effect coefficient indicating to what extent content of the healthcare review would have been caused by the therapy by use of therapy causation modeling based on a combination of the personal health records and electronic medical records of the patient, one or more staff experience records of one or more healthcare professionals providing the therapy, and one or more known therapy causation models, wherein the therapy causation modeling comprises profiling any time segments on effects of the therapy presented in a therapy causation model corresponding to the therapy.

9. The computer implemented method of claim 1,
wherein the step of ascertaining comprises determining that the identity of the author is the patient who received a therapy or a person involved with providing care to the patient;
wherein the step of validating further comprises determining a therapy effect coefficient indicating to what extent content of the healthcare review would have been caused by the therapy by use of therapy causation modeling based on a combination of the personal health records or electronic medical records of the patient, one or more staff experience records of one or more healthcare professionals providing the therapy, and one or more known therapy causation models, wherein the therapy causation modeling comprises analyzing the time progression of the subject, the respective times of the changes in a lifestyle, behavior, environment, or context of the patient, and the timeline of the therapy based on the time segments of the effect of the therapy.

10. The computer implemented method of claim 1,
wherein the step of ascertaining comprises determining that the identity of the author is the patient who received a therapy or a person involved with providing care to the patient;
wherein the step of validating further comprises determining a therapy effect coefficient indicating to what extent content of the healthcare review would have been caused by the therapy by use of therapy causation modeling based on a combination of the personal health records and electronic medical records of the patient, one or more staff experience records of one or more healthcare professionals providing the therapy, wherein the therapy causation modeling comprises:
profiling a time progression of the subject in the healthcare review, respective times of any changes in a lifestyle, behavior, environment, or context of the patient that can affect the therapy with respect to the time progression, a timeline of the therapy for the patient, any time segments on effects of the therapy presented in a therapy causation model corresponding to the therapy; and
analyzing the time progression of the subject, the respective times of the changes in the lifestyle, behavior environment, or context of the patient, and the timeline of the therapy based on the time segments of the effect of the therapy.

11. The computer implemented method of claim 1,
wherein the step of ascertaining comprises determining that the identity of the author is the patient who received a therapy or a person involved with providing care to the patient; and
wherein the step of validating comprises determining a therapy adherence by the patient indicating how well the patient participated in the therapy, a therapy effect coefficient indicating to what extent content of the healthcare review would have been caused by the therapy by use of therapy causation modeling, and a certainty indicator corresponding to the therapy causation coefficient indicating how statistically reliable the therapy coefficient is, based on a combination of the personal health records or electronic medical records of the patient, one or more staff experience records of one or more healthcare professionals providing therapy, one or more known therapy causation models.

12. The computer implemented method of claim 1,
wherein the step of ascertaining comprises determining that the identity of the author is the patient who received a therapy or a person involved with providing care to the patient;
wherein the step of validating further comprises:
determining a therapy adherence by the patient indicating how well the patient participated in the therapy, based on personal health records or electronic medical records of the patient and staff experience records of a healthcare professional providing the therapy to the patient;
determining a therapy effect coefficient indicating to what extent content of the healthcare review would have been caused by the therapy by use of therapy causation modeling based on a combination of the personal health records or electronic medical records of the patient, the staff experience records of the healthcare professional, and one or more known therapy causation models, wherein the therapy causation modeling comprises analyzing a time progression of the subject, respective times of the changes in a lifestyle, behavior environment, or context of the patient, and a timeline of the therapy based on the time segments of the effect of the therapy; and
determining a certainty indicator corresponding to the therapy causation coefficient indicating how statistically reliable the therapy coefficient is; and
wherein the step of determining the review validity score comprising:
combining the therapy adherence, the therapy causation coefficient, and the certainty indicator by use of a selected statistical method to indicate how valid the healthcare review by the patient is in view of all available data.

13. The computer implemented method of claim 1,
wherein the step of ascertaining comprises determining that the identity of the author is another healthcare professional representing an individual or an institute other than a healthcare professional providing therapy to the patient; and
wherein the step of validating comprises probing a relationship between the author and the healthcare professional, based on respective staff experience records of the author or an employee record of the author to see if the relationship is likely to bias the healthcare review.

14. The computer implemented method of claim 1,
wherein the step of ascertaining comprises determining that the identity of the author is another healthcare professional representing an individual or an institute other than a healthcare professional providing therapy to the patient; and
wherein the step of validating comprises evaluating the healthcare review based on standard operating procedures (SOP), care protocols, or guidelines regarding the therapy.

15. The computer implemented method of claim 1,
wherein the step of ascertaining comprises determining that the identity of the author is another healthcare professional representing an individual or an institute other than a healthcare professional providing therapy to the patient; and
wherein the step of validating further comprises substantiating the healthcare review based on electronic medical records corresponding to the therapy, data logs from local or remote computer applications used in the therapy, or data logs from medical equipment used in the therapy.

16. The computer implemented method of claim 1,
wherein the step of ascertaining comprises determining that the identity of the author is another healthcare professional representing an individual or an institute other than a healthcare professional providing therapy to the patient; and
wherein the step of determining the review validity score comprises combining the relationship between the author and the healthcare professional and respective results from the evaluating and the substantiating such that the review validity score is configured to represent whether the healthcare review is objectively trustworthy, whether the therapy in the healthcare review is in accord with a standard practice of the therapy, or whether the healthcare review is based on factual information corresponding to the patient or the therapy.

17. The computer implemented method of claim 1,
wherein the step of ascertaining comprises determining that the identity of the author is the patient who received a therapy or a person involved with providing the therapy to the patient; and
wherein the step of transmitting comprises ascertaining that the review validity score of the healthcare review satisfies a threshold configuration for adding to the staff experience record.

18. The computer implemented method of claim 1,
wherein the step of ascertaining comprises determining that the identity of the author is another healthcare professional representing an individual or an institute other than a healthcare professional providing therapy to the patient; and
wherein the step of transmitting comprises ascertaining that the review validity score of the healthcare review satisfies a threshold configuration for adding to the staff experience record.

19. A system comprising a memory, one or more processors in communication with the memory, and program instructions executable by the one or more processors via the memory configured to:
obtain from an author, a request to post a healthcare review to a staff experience record;
analyze the healthcare review to identify a subject of the healthcare review;
ascertain an identity of the author as to how the author is related with the subject;
validate the healthcare review based on the identity of the author, at least one of personal health records and electronic medical records corresponding to the author or the subject, and the staff experience record;
determine a review validity score of the healthcare review based on results from the validating of the healthcare review; and
transmit the healthcare review and the validity score to the staff experience record.

20. A computer program product comprising data representing program instructions executable by one or more processors via a memory configured to:
obtain from an author, a request to post a healthcare review to a staff experience record;
analyze the healthcare review to thereby identify a subject of the healthcare review;
ascertain an identity of the author as to how the author is related with the subject;
validate the healthcare review regarding factual basis and objectivity of content based on the identity of the author, at least one of personal health records and electronic medical records corresponding to the author or the subject, and the staff experience record;
determine a review validity score of the healthcare review based on results from the validating of the healthcare review; and
transmit the healthcare review and the validity score to the staff experience record.
